(19) United States
(12) Patent Application Publication
(10) Pub. No.: US 2020/0030377 A1
(43) Pub. Date: Jan. 30, 2020

(54) ANTIGEN-SPECIFIC IMMUNE MODULATION

(71) Applicant: The Curators of the University of Missouri, Columbia, MO (US)

(72) Inventor: Habib Zaghouani, Columbia, MO (US)

(21) Appl. No.: 16/335,694

(22) PCT Filed: Sep. 22, 2017
(86) PCT No.: PCT/US2017/052849

§ 371 Date: Mar. 22, 2019

Related U.S. Application Data
(60) Provisional application No. 62/398,972, filed on Sep. 23, 2016.

(51) Int. Cl. A61K 35/17; A61P 3/10; C07K 16/28

(52) U.S. Cl. CPC A61K 35/17; C07K 2317/76; C07K 16/2827; A61P 3/10

(57) ABSTRACT

The disclosure relates generally to methods of treating or preventing an autoimmune disease, for example diabetes, by activating PD-L1 in an antigen presenting cell.

### PRIORITY CLAIM

This application is a 35 U.S.C. § 371 U.S. National Stage Application of International Application No. PCT/US2017/052849 filed Sep. 22, 2017, which claims priority to U.S. Provisional Patent Application Ser. No. 62/398,972 which was filed on Sep. 23, 2016, the entire contents of which are incorporated herein by reference and relied upon.

### STATEMENT REGARDING FEDERAL FUNDING

This technology was made with government support under Grant No. RO1DK093515 awarded by National Institutes of Health. The government has certain rights in the invention.

### FIELD

The present technology provides methods of treating autoimmune diseases. In particular, provided herein are modified antigen-presenting cells that impair or kill T cells.

### BACKGROUND

Autoimmune diseases occur when one's immune system mistakes its own cells as foreign invaders and attacks and kills the body's own healthy cells. Autoimmune diseases can affect almost any part of the body, including the heart, brain, nerves, muscles, skin, eyes, joints, lungs, kidneys, glands, the digestive tract, and blood vessels. Many autoimmune diseases affect multiple parts of the body. The National Institutes of Health has estimated that approximately 23.5 million Americans suffer from autoimmune diseases and that the prevalence is rising. The causes and triggers of many autoimmune diseases are unknown but it is believed that often a combination of genetic tendencies and environmental factors under the right conditions may trigger an autoimmune response. However, many of these diseases lack adequate treatments.

As such, there remains a need for improved method and compositions for the treatment of autoimmune diseases.

### SUMMARY

In some aspects provided herein are methods of treating or preventing an autoimmune disease in a subject in need thereof comprising, administering to the subject an effective amount of modified antigen-presenting cells (APCs) having increased expression of programmed death ligand 1 (PD-L1).

In some aspects provided herein are compositions comprising a modified APC having increased expression of programmed death ligand 1 (PD-L1).

In some embodiments, the increased expression of PD-L1 is induced by contacting the APC with an immunoglobulin, for example, an immunoglobulin-polypeptide chimera.

In some embodiments, the methods further comprise isolating APCs from the subject or an APC donor to obtain purified APCs. In some embodiments, the methods further comprise contacting the purified APCs with a targeting molecule, for example, immunoglobulin-polypeptide chimera, to generate modified APCs.

In some embodiments, the immunoglobulin-polypeptide chimera is selected from the group consisting of Ig-GAD2, Ig-GAD1, Ig-p79, Ig-INSβ, and any combination thereof.

In some embodiments, the methods further comprise administering to the subject an effective amount of an immunoglobulin, for example an immunoglobulin-polypeptide chimera, prior to isolating the APCs.

In other embodiments, at least a portion of the increased expression of PD-L1 is from overexpression of exogenous PD-L1.

In some embodiments, the APC is a dendritic cell, a macrophage, a Langerhans cell, a B cell, or a combination thereof.

In some embodiments, the modified APCs are characterized by one or more of the following characteristics: (i) no change or a non-significant change in expression of at least one stimulatory molecule; (ii) an ability to suppress inflammatory cytokine production; (iii) an ability to increase anti-inflammatory cytokine production; (iv) an ability to downregulate mTORR expression; (v) an ability to inhibit activation of a T cell; (vi) an ability to kill a T cell; (vii) an ability to energized self-reactive T cells; and (viii) an ability to inhibit the trafficking of self-reactive T cells, as compared to a control, such as, an APC that does not have increased expression of PD-L1.

In some embodiments, the at least one stimulatory molecule is CD80, CD86, or both. In some embodiments, the inflammatory cytokine is selected from the group consisting of interleukin (IL)-1, IL-2, IL-9, IL-12, IL-17, IL-18IL-21, IL22, interferon-γ (IFN-γ, granulocyte-macrophage colony stimulating factor, and tumor necrosis factor-α (TNF-α). In some embodiments, the anti-inflammatory cytokine is selected from the group consisting of IL-4, IL-5, IL-10, IL-13, IFN-α, and transforming growth factor-β (TGF-β).

In some embodiments, the autoimmune disease is selected from the group consisting of a cardiovascular disease, a rheumatoid disease, a glandular disease, a gastrointestinal disease, a cutaneous disease, a hepatic disease, a neurological disease, a muscular disease, a nephric disease, a disease related to reproduction, a connective tissue disease, or a systemic disease. In some embodiments, the glandular disease is Type 1 diabetes.

In some embodiments, the compositions further comprise a pharmaceutically acceptable carrier/excipient.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. Furthermore, components can be shown as transparent in certain views for clarity of illustration only and not to indicate that the illustrated component is necessarily transparent. For ease of reference, throughout this disclosure identical reference numbers may be used to identify identical or at least generally similar, analogous and/or complementary components or features.

FIG. 1 demonstrates that Ig-p79-induced mTOR-mediated T cell tolerance correlates with PD-L1 upregulation on splenic APCs. (A and B) BDC2.5 Th1 cells were transferred into NOD.scid mice and the hosts were treated with Ig-p79 or Ig-HEL. FIG. 1A shows expression of CD80, CD86, PD-L1, and PD-L2 on splenic CD3²F4/80²CD11c⁺ dendritic cells (DCs) 1 day after Ig chimera treatment. Results are representative of three independent experiments. FIG. 1B shows MFI of PD-L1 on splenic DCs, macrophages (CD11b⁺F4/80⁺), and B cells (CD19⁺B220⁺) from three independent experiments performed as described in FIG. 1A. Each bar represents the mean 6 SEM of three experiments. FIG. 1C shows NOD.scid mice (Nil+Ig-p79) and NOD.scid mice recipient of BDC2.5 Th1 cells (T cells+Ig-p79) were treated with Ig-p79, and 1 day later PD-L1 expression was examined on CD3²F4/80²CD11c′ splenic DCs. The bar graph shows the mean 6 SEM MFI from four independent experiments. FIG. 1D depicts splenic DCs from Ig-p79 (DC_{Ig-p79})- or Ig-HEL (DC_{Ig-HEL})-treated NOD.scid mice recipient of BDC2.5 Th1 cells were sorted on the basis of CD11c expression with exclusion of CD3⁺ cells. The DC_{Ig-p79} and DC_{Ig-HEL} cells were loaded with free p79 peptide and cultured with naive BDC2.5 CD4⁺ CD25² T cells for 72 h. IFN-g production was measured by ELISA. A group of p79-loaded DC_{Ig-p79} coated with anti-PD-L1 Ab was included for control purposes. Each bar represents the mean 6 SEM of three independent experiments. **p, 0.01. FIG. 1E depicts NOD.scid mice recipient of BDC2.5 Th1 cells treated with Ig-p79 or Ig-HEL, and 7 d later the expression of PD-1 by splenic (upper panel) and pancreatic (lower panel) T cells was analyzed by flow cytometry. Results are representative of three independent experiments. The numbers on top of the panels represent the MFI obtained upon treatment with Ig-HEL/Ig-p79, respectively. The insets represent the mean 6 SEM of MFI collected from three independent experiments. *p, 0.05,**p, 0.01.

FIG. 2 is demonstrates that a blockade of PD-L1 restores mTORC1 activity and nullifies Ig-p79-induced tolerance. NOD.scid mice (six per group) were given BDC2.5 Th1 cells and treated with Ig-p79 alone or together with anti-PD-L1 Ab (200 mg/mouse, i.p.). Another injection of anti-PD-L1 Ab was given on days 3 and 5 after Ig-p79 treatment. FIG. 2A shows diabetes incidence up to day 16 after BDC2.5 transfer (n=6 per group). FIG. 2B shows a representative plot of CD3⁺CD4⁺ T cells in spleens and pancreas on day 16 post-BDC2.5 Th1 cell transfer. FIG. 2C shows the mean 6 SEM of the percentage (left panel) and absolute numbers (right panel) of CD3⁺CD4⁺ T cells compiled from three independent experiments. FIG. 2D shows S6 phosphorylation (pS6) and CXCR3 and T-bet expression in splenic and pancreatic CD3⁺CD4⁺ T cells on day 16 post-BDC2.5 Th1 transfer. Results are representative of three independent experiments. *p, 0.05, **p, 0.01, ***p, 0.001. n.s., not significant

### DETAILED DESCRIPTION

The present technology is directed to methods of treating an autoimmune disease. To contain autoimmunity, pathogenic T cells must be eliminated or diverted from reaching the target organ. The present disclosure defines the signaling events underlying antigen-induced (Ag-induced) chemokine receptor-mediated tolerance. Specifically, the mammalian target of rapamycin complex 1 (mTORC1) is shown to be is a major target for induction of CXCR3 downregulation and crippling of Th1 cells. Indeed, Ag administration induces upregulation of programmed death-ligand 1 on dendritic cells in a T cell-dependent manner. In return, programmed death-ligand 1 interacts with the constitutively expressed programmed death-1 on the target T cells and stimulates docking of Src homology 2 domain-containing tyrosine phosphatase 2 phosphatase to the cytoplasmic tail of programmed death-1. Active Src homology 2 domain-containing tyrosine phosphatase 2 impairs the signaling function of the PI3K/protein kinase B (AKT) pathway, leading to functional defect of mTORC1, downregulation of CXCR3 expression, and suppression of T1D. Thus, the disclosure demonstrates that the mTORC1 component of the metabolic pathway serves as a target for chemokine receptor-mediated T cell tolerance and suppression of T1D.

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about.” It also is to be understood, although not always explicitly stated, that the reagents described herein are merely exemplary and that equivalents of such are known in the art.

It must be noted that as used herein and in the appended claims, the singular forms “a”, “an”, and “the” include plural referents unless the context clearly dictates otherwise. Thus, for example, reference to “an antigen presenting cell” includes a plurality of antigen presenting cells. As used herein the following terms have the following meanings.

The term “about” when used before a numerical designation, e.g., temperature, time, amount, concentration, and such other, including a range, indicates approximations which may vary by (+) or (−) 20%, 10%, 5% or 1%.

Also as used herein, “and/or” refers to and encompasses any and all possible combinations of one or more of the associated listed items, as well as the lack of combinations when interpreted in the alternative (“or”).

“Comprising” or “comprises” is intended to mean that the compositions of, for example, cells and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The terms “administering,” “administer” and the like refer to introducing an agent (e.g., an APC and/or immunoglobulin) into a subject. Typically, an effective amount is administered, which amount can be determined by the treating physician or the like. Any route of administration, such as topical, subcutaneous, peritoneal, intravenous, intraarterial, inhalation, vaginal, rectal, nasal, buccal, introduction into the cerebrospinal fluid, or instillation into body compartments can be used. The terms and phrases “administering” and “administration of,” when used in connection with a composition (and grammatical equivalents) refer both to direct administration, which may be administration to a patient by a medical professional or by self-administration by the patient, and/or to indirect administration, which may be the act of prescribing a drug. For example, a physician who instructs a patient to self-administer an agent (e.g., an APC and/or immunoglobulin) and/or provides a patient with a prescription for a drug is administering the agent to the patient. “Periodic administration” or “periodically administering” refers to multiple treatments that occur on a daily, weekly, or a monthly basis. Periodic administration may also refer to administration of an agent one, two, three or more time(s) per day.

As used herein, the term “autoimmune disease” refers to a disease where the body's immune system attacks its own cells or tissues. Autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

An “effective amount” is an amount of an agent or compound (e.g., antigen-presenting cells) sufficient to effect beneficial or desired results. An effective amount can be in one or more administrations, applications or dosages. Determination of these parameters is well within the skill of the art. These considerations, as well as effective formulations and administration procedures are well known in the art and are described in standard textbooks.

The term “isolated” as used herein with respect to cells, nucleic acids, such as DNA or RNA, refers to molecules separated from other DNAs or RNAs, respectively that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. An “isolated cell,” for example, an isolated antigen presenting cell is a cell that is substantially free of other cellular material, tissue, medium of the environment in which it is naturally found.

A “subject,” “individual” or “patient” is used interchangeably herein and refers to a vertebrate, for example a primate, a mammal or preferably a human. Mammals include, but are not limited to equines, canines, bovines, ovines, murines, rats, simians, humans, farm animals, sport animals and pets.

### Autoimmune Diseases

Provided herein are compositions and methods for treating autoimmune diseases. In some embodiments, the autoimmune disease is an autoimmune cardiovascular disease. Non-limiting examples of autoimmune cardiovascular diseases include atherosclerosis, myocardial infarction, thrombosis, Wegener's granulomatosis, Takayasu's arteritis, Kawasaki syndrome, anti-factor VIII autoimmune disease, necrotizing small vessel vasculitis, microscopic polyangiitis, Churg and Strauss syndrome, pauci-immune focal necrotizing and crescentic glomerulonephritis, antiphospholipid syndrome, antibody-induced heart failure, thrombocytopenic purpura, autoimmune hemolytic anemia, cardiac autoimmunity in Chagas' disease, and anti-helper T lymphocyte autoimmunity.

In some embodiments, the autoimmune disease is an autoimmune rheumatoid disease. Non-limiting examples of autoimmune rheumatoid diseases include rheumatoid arthritis and ankylosing spondylitis.

In some embodiments, the autoimmune disease is an autoimmune glandular disease. Non-limiting examples of autoimmune glandular diseases include pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome.

In some embodiments, the autoimmune disease is an autoimmune gastrointestinal disease. Non-limiting examples of autoimmune gastrointestinal diseases include chronic inflammatory intestinal diseases, celiac, inflammatory bowel disease (IBD) including Crohn's disease, ileitis and ulcerative colitis.

In some embodiments, the autoimmune disease is an autoimmune cutaneous disease. Non-limiting examples of autoimmune cutaneous diseases include autoimmune bullous skin diseases, such as, pemphigus vulgaris, bullous pemphigoid and pemphigus foliaceus. Examples of autoimmune hepatic diseases include, but are not limited to, hepatitis, autoimmune chronic active hepatitis, primary biliary cirrhosis, and autoimmune hepatitis.

In some embodiments, the autoimmune disease is an autoimmune neurological disease. Non-limiting examples of autoimmune neurological diseases include multiple sclerosis, Alzheimer's disease, myasthenia gravis, neuropathies, motor neuropathies, Guillain-Barre syndrome and autoimmune neuropathies, myasthenia, Lambert-Eaton myasthenic syndrome, paraneoplastic neurological diseases, cerebellar atrophy, paraneoplastic cerebellar atrophy and stiff-man syndrome, non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies, dysimmune neuropathies, acquired neuromyotonia, arthrogryposis multiplex congenital, neuritis, optic neuritis, and neurodegenerative diseases.

In some embodiments, the autoimmune disease is an autoimmune muscular disease. Non-limiting examples of autoimmune muscular diseases include myositis, autoimmune myositis and primary Sjogren's syndrome, and smooth muscle autoimmune disease.

In some embodiments, the autoimmune disease is an autoimmune nephric disease. Non-limiting examples of autoimmune nephric diseases include nephritis and autoimmune interstitial nephritis.

In some embodiments, the autoimmune disease is an autoimmune connective tissue disease. A non-limiting example of an autoimmune connective tissue disease is autoimmune ear disease.

In some embodiments, the autoimmune disease is an autoimmune systemic disease. Non-limiting examples of autoimmune systemic diseases include systemic lupus erythematosus and systemic sclerosis.

### Antigen-Presenting Cells

Provided herein are modified antigen-presenting cells (APC) for use in treating an autoimmune disease. The APC can be any suitable APC known to one of skill in the art. For example, the APC can be a dendritic cell, a macrophage, a Langerhans cell, or a B cell, or any combination thereof. In some embodiments, the APC is a dendritic cell.

In some embodiments, the methods involve isolating APCs from the subject or an APC donor to obtain purified APCs. Accordingly, the APCs can be autologous APCs or allogeneic APCs. The APCs can be isolated by any method known to one of skill in the art. In some embodiments, the APCs are isolated based on the presence of a particular marker or combination of markers including, for example, CD8, CD8alpha, CD11b, Cd11c, CD103, CD205, CD24, CD115, CD117, CD135, CD11c^{low} CD45RA, CD123, ILT-7, MHC class II, MHC Class II^{low}, TLR7, and/or TRL9. In some embodiments, the APCs are isolated by cell sorting for Cd11c. In some embodiments, the APCs are isolated based on the absence of a particular marker, for example, CD3, CD14, CD19, CD56, and/or CD66b.

In some embodiments, after isolating the APC the APC is modified to express a targeting molecule to generate a modified APC. For example, the targeting molecule can be an an immunoglobulin-polypeptide chimera. In some embodiments, the immunoglobulin-polypeptide chimera is selected from the group consisting of Ig-GAD2, Ig-GAD1, Ig-p79, and any combination thereof. WO 2014/138725 describes several suitable immunoglobulin-polypeptide chimeras and is incorporated by reference in its entirety. In some embodiments, multiple immunoglobulin-polypeptides are used to target multiple epitopes to treat the same or different autoimmune disease. In some embodiments, the methods comprise administering to the subject an effective amount of an immunoglobulin prior to isolating the APCs. The immunoglobulin can be administered by any appropriate route, which will be apparent to the skilled person depending on the disease or condition to be treated. Typical routes of administration include intravenous, intra-arterial, intramuscular, subcutaneous, intracranial, intranasal or intraperitoneal. In other embodiments, the APCs are modified to express exogenous PD-L1, for example, the exogenous PD-L1 can be a nucleic acid sequence encoding PD-L1. Any modification method known to one of skill in the art can be employed to express exogenous PD-L1, including viral vectors (e.g., adenoviruses, retroviruses, particularly replication defective retroviruses, and lentiviruses) or non-viral vectors (e.g., episomal). In some embodiments, the PD-L1 is a synthetic messenger RNA (mRNA). Synthetic mRNAs provide the genetic information for making proteins of interest and can be chemically modified to avoid triggering an immune response. Zangi et al. (2013) Nature Biotech 31:898-907. Since mRNAs do not integrate into the host cell genome, the synthetic RNA acts for a period of time and then disappears as the cell divides. In some embodiments the synthetic mRNAs are modified, for example, with pseudouridine and/or 5-methyl-cytidine, to reduce innate antiviral response to single-stranded RNA. In some embodiments, the synthetic RNAs encode PD-L1 and/or equivalents of each thereof.

In some embodiments, the exogenous PD-L1 is introduced into an APC using any one of a variety of well-known techniques, such as non-viral based transfection of the cell. Introduction into the APC may be performed by any non-viral based transfection method known in the art, such as, but not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like, or cell fusion. Other methods of transfection include transfection reagents such as Lipofectamine™, Dojindo Hilymax™, Fugene™, jetPEI™, Effectene™, and DreamFect™.

In one embodiment, the subject is injected with an immunoglobulin, for example, Ig-GAD2 to upregulate PD-L1 in vivo. The APCs are then isolated from the subject and modified to have a targeting molecule, for example, Ig-GAD2, Ig-p79, peptide, or mix of peptides. Then the modified APCs are reinjected into the patient to treat the autoimmune disease. In another embodiment, the APCs are isolated from a subject (or donor subject) and modified to have a targeting molecule, for example, Ig-GAD2, Ig-p79, or Ig-INSβ peptide, or mix of peptides. The modified APCs are then injected into a patient to treat an autoimmune disease, wherein it is contemplated that the peptide or other target-presenting APC will interact with T cells and upregulate PD-L1 in vivo. In yet another embodiment, the APCs are isolated from a subject (or donor subject) and modified to have a targeting molecule, for example, Ig-GAD2, Ig-p79, or Ig-INSβ peptide, or mix of peptides. The APCs are then further modified to overexpress PD-L1. The modified APCs are then injected into a patient to treat an autoimmune disease.

In some embodiments, the methods comprise administering to the subject an effective amount of the modified APCs. The modified APCs can be administered by any appropriate route, which will be apparent to the skilled person depending on the disease or condition to be treated. Typical routes of administration include intravenous, intra-arterial, intramuscular, subcutaneous, intracranial, intranasal or intraperitoneal.

In some embodiments, about 1×10⁸ to about 1×10¹¹ cells per m² of body surface area of the subject are administered to the subject. The APCs can be administered to an individual by absolute numbers of cells, e.g., said individual can be administered from about 1000 cells/injection to up to about 10 billion cells/injection, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) APCs per injection, or any ranges between any two of the numbers, end points inclusive.

In other embodiments, the subject can be administered from about 1000 cells/injection/m² to up to about 10 billion cells/injection/m², such as at about, at least about, or at most about, 1×10⁸/m², 1×10⁷/m², 5×10⁷/m², 1×10⁶/m², 5×10⁶/m², 1×10⁵/m², 5×10⁵/m², 1×10⁴/m², 5×10⁴/m², 1×10³/m², 5×10³/m² (and so forth) APCs per injection, or any ranges between any two of the numbers, end points inclusive.

In other embodiments, APCs can be administered to such individual by relative numbers of cells, e.g., said individual can be administered about 1000 cells to up to about 10 billion cells per kilogram of the individual, such as at about, at least about, or at most about 1×10⁸, 5×10⁷, 1×10⁷, 5×10⁶, 1×10⁶, 5×10⁵, 1×10⁵, 5×104, 1×10⁴, 5×10³, 1×10³, (and so forth) APCs per kilogram of the individual, or any ranges between any two of the numbers, end points inclusive.

The APCs, and optionally other autoimmune therapies can be administered once to a patient with an autoimmune disease or can be administered multiple times, e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10 or more weeks during therapy, or any ranges between any two of the numbers, end points inclusive.

In another embodiment, upon treatment with the APCs of the present dislcosure, the subject or subject group exhibits one or more of the following outcomes:

(i) no change or a non-significant change in expression of at least one stimulatory molecule (e.g., CD80 and CD86);

(ii) suppression in inflammatory cytokine production of at least about 5%, at least about 10%, at least about 15%, at least about 20%, at least about 25%, at least about 30%, at least about 35%, at least about 40%, at least about 45%, at least about 50%, at least about 55%, at least 60%, at least 65%, at least 70%, at least about 75%, at least 80%, at least 85%, at least 90%, at least 95%, or at least 99% (actual % change or median % change) as compared to a control;

(iii) an increase anti-inflammatory cytokine production of at least about 5%, at least about 10%, at least about 15%, at least about 20%, at least about 25%, at least about 30%, at least about 35%, at least about 40%, at least about 45%, at least about 50%, at least about 55%, at least 60%, at least 65%, at least 70%, at least about 75%, at least 80%, at least 85%, at least 90%, at least 95%, or at least 99% (actual % change or median % change) compared to a control;

(iv) a reduction in mTOR expression/signaling of at least about 5%, at least about 10%, at least about 15%, at least about 20%, at least about 25%, at least about 30%, at least about 35%, at least about 40%, at least about 45%, at least about 50%, at least about 55%, at least 60%, at least 65%, at least 70%, at least about 75%, at least 80%, at least 85%, at least 90%, at least 95%, or at least 99% (actual % change or median % change) as compared to a control;

(v) inhibition of T cell activation of at least about 5%, at least about 10%, at least about 15%, at least about 20%, at least about 25%, at least about 30%, at least about 35%, at least about 40%, at least about 45%, at least about at least about 50%, at least about 55%, at least 60%, at least 65%, at least 70%, at least about 75%, at least 80%, at least 85%, at least 90%, at least 95%, or at least 99% (actual % change or median % change) as compared to a control;

(vi) an ability to kill a T cell;

(vii) energize self-reactive T cells by at least about 5%, at least about 10%, at least about 15%, at least about 20%, at least about 25%, at least about 30%, at least about 35%, at least about 40%, at least about 45%, at least about 50%, at least about 55%, at least 60%, at least 65%, at least 70%, at least about 75%, at least 80%, at least 85%, at least 90%, at least 95%, or at least 99% (actual % change or median % change) as compared to a control; and/or

(viii) an ability to inhibit the trafficking of self-reactive T cells by least about 5%, at least about 10%, at least about 15%, at least about 20%, at least about 25%, at least about 30%, at least about 35%, at least about 40%, at least about 45%, at least about 50%, at least about 55%, at least 60%, at least 65%, at least 70%, at least about 75%, at least 80%, at least 85%, at least 90%, at least 95%, or at least 99% (actual % change or median % change) as compared to a control.

The control can be a subject treated with a placebo, a baseline control, or a subject treated with unmodified APCs.

In some embodiments, the inflammatory cytokine is selected from the group consisting of interleukin (IL)-1, IL-2, IL-9, IL-12, IL-17, IL-18IL-21, IL22, interferon-γ (IFN-γ, granulocyte-macrophage colony stimulating factor, and tumor necrosis factor-α (TNF-α).

In some embodiments, the anti-inflammatory cytokine is selected from the group consisting of IL-4, IL-5, IL-10, IL-13, IFN-α, and transforming growth factor-β (TGF-β).

In some embodiments, the modified APCs are administered to the subject for a period effective to reduce at least one symptom of an autoimmune disease by at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30% at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, at least about 98%, at least about 99%, or by 100% compared to a control. The control can be a subject treated with a placebo, a baseline control, or a subject treated with an unmodified APCs.

Non-limiting symptoms include, inflammation of joints and surrounding tissues, a decrease in red blood cell count, white patches on skin, redness and/or irritation of the skin, inflammation of glands or organs (e.g., thyroid gland and small intestine), overactive thyroid gland, hormone dysregulation, dry eye, loss of muscle mass and/or tone, loss of neural cells, and destruction of insulin-producing cells.

In some embodiments the modified APCs are administered with at least one other autoimmune therapy. Suitable other autoimmune therapies include any autoimmune therapy known to one of skill in the art. Non-limiting examples of other autoimmune therapies include, probiotics, immunosuppressives (e.g., corticosteroids), etanercept, infliximab, prednisone, and azathioprine. In some embodiments, the modified APCs and the at least one other autoimmune therapy are administered simultaneously. In other embodiments, the modified APCs and the at least one other autoimmune therapy are administered sequentially.

### Compositions

In some embodiments, the present disclosure provides a composition comprising at least one of the modified APCs described herein. In some embodiments, the composition further comprises a pharmaceutically acceptable excipient, diluent, carrier, or any combination thereof. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The composition may comprise a pharmaceutically acceptable excipient, a pharmaceutically acceptable salt, diluents, carriers, vehicles and such other inactive agents well known to the skilled artisan. Vehicles and excipients commonly employed in pharmaceutical preparations include, for example, talc, gum Arabic, lactose, starch, magnesium stearate, cocoa butter, aqueous or non-aqueous solvents, oils, paraffin derivatives, glycols, etc. Solutions can be prepared using water or physiologically compatible organic solvents such as ethanol, 1,2-propylene glycol, polyglycols, dimethylsulfoxide, fatty alcohols, triglycerides, partial esters of glycerine and the like. Compositions may be prepared using conventional techniques that may include sterile isotonic saline, water, 1,3-butanediol, ethanol, 1,2-propylene glycol, polyglycols mixed with water, Ringer's solution, etc. In one aspect, a coloring agent is added to facilitate in locating and properly placing the composition to the intended treatment site.

Compositions may include a preservative and/or a stabilizer. Non-limiting examples of preservatives include methyl-, ethyl-, propyl-parabens, sodium benzoate, benzoic acid, sorbic acid, potassium sorbate, propionic acid, benzalkonium chloride, benzyl alcohol, thimerosal, phenylmercurate salts, chlorhexidine, phenol, 3-cresol, quaternary ammonium compounds (QACs), chlorbutanol, 2-ethoxyethanol, and imidurea.

To control tonicity, the composition can comprise a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride and calcium chloride.

Compositions may include one or more buffers. Typical buffers include: a phosphate buffer; a Tris buffer; a borate buffer; a succinate buffer; a histidine buffer; or a citrate buffer. Buffers will typically be included at a concentration in the 5-20 mM range. The pH of a composition will generally be between 5 and 8, and more typically between 6 and 8 e.g. between 6.5 and 7.5, or between 7.0 and 7.8.

In some embodiments, the composition may include a cryoprotectant agent. Non-limiting examples of cryoprotectant agents include a glycol (e.g., ethylene glycol, propylene glycol, and glycerol), dimethyl sulfoxide (DMSO), formamide, sucrose, trehalose, dextrose, and any combinations thereof.

In one embodiment, the cell is part of a population of cultured cells (i.e., in vitro). In another embodiment, the cell is part of a population of cells of a subject (i.e., in vivo). For example, the targeting molecule (e.g., immunoglobulin-polypeptide chimera) may be delivered to an in vivo cell or an in vivo population of cells that form a tissue or organ in a subject for the purpose of treating or preventing an autoimmune disease. Alternatively, the targeting molecule (e.g., immunoglobulin-polypeptide chimera) may be delivered to a cultured cell or a population of cultured cells for the purpose of conducting experiments to study its effect on a particular type of cell.

The composition can be included in an implantable device. Suitable implantable devices contemplated by this invention include intravascular stents (e.g., self-expandable stents, balloon-expandable stents, and stent-grafts), scaffolds, grafts, and the like. Such implantable devices can be coated on at least one surface, or impregnated, with a composition capable of treating or preventing an autoimmune disease, for example Type 1 diabetes.

The compositions can be administered to a subject by any suitable mode and route. Non-limiting examples include internal, pulmonary, rectal, nasal, vaginal, lingual, intravenous, intraarterial, intramuscular, intraperitoneal, intracutaneous and subcutaneous routes. Compositions may also be suitable for transdermal delivery as part of a cream, gel, or patch. Other dosage forms include tablets, capsules, pills, powders, aerosols, suppositories, parenterals, and oral liquids, including suspensions, solutions and emulsions. Sustained release dosage forms may also be used.

### EXAMPLES

### Example 1

### APCs Upregulate PD-L1 upon Interaction with T Cells Undergoing mTOR Mediated Ag-Specific Tolerance

T cells must recognize their cognate Ag presented by competent APCs expressing optimal costimulatory molecules to undergo activation (Mueller, D. L., M. K. Jenkins, and R. H. Schwartz. 1989. Clonal expansion versus functional clonal inactivation: a costimulatory signalling pathway deter-mines the outcome of T cell antigen receptor occupancy. Annu. Rev. Immunol. 7: 445-480). In contrast, when the Ag is delivered to APCs under noninflammatory conditions that drive presentation with minimal costimulation, tolerance rather than activation ensues (Legge, K. L., B. Min, J. J. Bell, J. C. Caprio, L. Li, R. K. Gregg, and H. Zaghouani. 2000. Coupling of peripheral tolerance to endogenous interleukin 10 promotes effective modulation of myelin-activated T cells and ameliorates experimental allergic encephalomyelitis. J. Exp. Med. 191: 2039-2052 and Legge, K. L., R. K. Gregg, R. Maldonado-Lopez, L. Li, J. C. Caprio, M. Moser and H. Zaghouani. 2002. On the role of dendritic cells in peripheral T cell tolerance and modulation of autoimmunity. J. Exp. Med. 196: 217-227). The costimulatory network is diverse and includes molecules on the surface of APCs and their ligands on T cells (Greenwald, R. J., G. J. Freeman, and A. H. Sharpe. 2005. The B7 family revisited. Annu. Rev. Immunol. 23: 515-548, Fife, B. T., and J. A. Bluestone. 2008. Control of peripheral T-cell tolerance and autoimmunity via the CTLA-4 and PD-1 pathways. Immunol. Rev. 224: 166-182, and Chen, L. 2004. Co-inhibitory molecules of the B7-CD28 family in the control of T-cell immunity. Nat. Rev. Immunol. 4: 336-347).

To determine which of these costimulatory molecules on APCs target mTORC1 to drive T cell tolerance, APCs were assessed for alteration in the expression of inhibitory (PD-L1 and PD-L2) as well as stimulatory (CD80, CD86) molecules. Briefly, and as described in detail in Chen, E., X. Wan, T. K. Ukah, M. M. Miller, S. Barik, A. Cattin-Roy, and H. Zaghouani. 2016. Antigen-Specific Immune Modulation Targets mTORC1 Function to Drive Chemokine Receptor-Mediated T Cell Tolerance. J. Immunol. 197(9):3554-3565 which is incorporated by reference in its entirety), NOD (H-2g7), NOD.scid, and NOD.BDC2.5 mice were purchased from The Jackson Laboratory (Bar Harbor, Me.). NOD.BDC2.5.GFP was generated by breeding BDC2.5 mice to NOD mice expressing GFP under the b-actin promoter (16). All mice were used at the age of 6-8 wk, according to the guidelines of the University of Missouri Animal Care and Use Committee. The p79 peptide corresponds to a library-defined mimotope (AVRPLWVRME), and hen egg lysosome (HEL) peptide corresponding to aa residues 11-25 of HEL was previously described (Wan, X., F. B. Guloglu, A. M. VanMorlan, L. M. Rowland, R. Jain, C. L. Haymaker, J. A. Cascio, M. Dhakal, C. M. Hoeman, D. M. Tartar, and H. Zaghouani. 2012. Mechanisms underlying antigen-specific tolerance of stable and convertible Th17 cells during suppression of autoimmune diabetes. Diabetes 61: 2054-2065). Ig-p79-expressing p79 mimotope and Ig-HEL-incorporating HEL peptide within the H chain V region have been previously described (Wan, X., F. B. Guloglu, A. M. VanMorlan, L. M. Rowland, R. Jain, C. L. Haymaker, J. A. Cascio, M. Dhakal, C. M. Hoeman, D. M. Tartar, and H. Zaghouani. 2012. Mechanisms underlying antigen-specific tolerance of stable and convertible Th17 cells during suppression of autoimmune diabetes. Diabetes 61: 2054-2065). Large cell culture production and affinity chromatography purification of Ig-p79 and Ig-HEL were accomplished, as described (Wan, X., F. B. Guloglu, A. M. VanMorlan, L. M. Rowland, R. Jain, C. L. Haymaker, J. A. Cascio, M. Dhakal, C. M. Hoeman, D. M. Tartar, and H. Zaghouani. 2012. Mechanisms underlying antigen-specific tolerance of stable and convertible Th17 cells during suppression of autoimmune diabetes. Diabetes 61: 2054-2065). The following Abs were used: mTOR mAb (7C10), phospho-S6 (Ser235/236) (D57.2.2E)-allophycocyanin, phospho-p70 S6 kinase (Thr389) (108D2) mAb, phosphor-AKT (Thr308) (C31E5E) mAb, phospho-Akt (Ser473) (D9E) mAb, GFP (D5.1) mAb, and SHP-2 (D50F2) mAb (Cell Signaling Tech-nology); allophycocyanin-conjugated donkey anti-rabbit IgG (Jackson ImmunoResearch); CD3e (145-2C11)-FITC, CD4 (RM4-5)-PE-Cy7, Vb4 (KT4)-PE, CD11c-allophycocyanin, CD11b-PE-Cy7, B220-FITC, CD19-PE-Cy7, and PD-L1-PE (BD Biosciences); F4/80-PE and PD-1-PE-Cy7 (BioLegend); CD71-allophycocyanin and CD98-PE, CD90.1 (Thy-1.1)-PE, CXCR3-allophycocyanin, CD80-PE, CD86-PE, and PD-L2-PE (eBioscience); PD-1 (J43) Ab (Novus); and T-bet-FITC and SH-PTP2 (C-18) (Santa Cruz Biotechnology).

Splenic cells from 4- to 6-wk-old naive NOD.BDC2.5 mice were polarized to Th1 cells, as follows: the cells (2 3 106 cells/ml) were stimulated with p79 peptide (0.5 mM) for 4 d in the presence of rIL-12 (10 ng/ml; PeproTech) and anti-IL-4 Ab (10 mg/ml). Polarized CD4⁺ Th1 cells were purified by negative selection using CD4 T Cell Isolation Kit II (Miltenyi Biotec) and stimulated with PMA (50 ng/ml) and ionomycin (500 ng/ml) for 2 h. The cells were then labeled using the mouse IFN-g (allophycocyanin) detection kit from Miltenyi Biotec. Subsequently, the enriched Th1 cells (IFN-g⁺) were sorted to at least 98% purity on a Beckman Coulter MoFlo XDP sorter and transferred i.v. into NOD.scid (6×10⁶ cells per mouse). Mice were monitored for T1D by measuring blood glucose level daily. A mouse is considered diabetic when blood glucose level is ≥300 mg/dl on two consecutive measurements.

For staining of surface Ags, cells (1×10⁷ cells per well) were incubated with mouse IgG (Sigma-Aldrich) to block FcγRs and stained with saturating concentrations of fluorochrome-labeled Ab in PBS containing 0.5% BSA. Dead cells were excluded using 7-amino-actinomycin D (EMD Biosciences). For intracellular staining, cells were stained for surface markers, treated with Fix/Perm buffer (eBioscience), and then incubated with conjugated Ab for intracellular staining; dead cells were excluded using fixable viability dye (eBioscience). Data were acquired on Beckman Coulter Cyan (Brea, Calif.) and analyzed using Flowjo 10.0.8 (Tree Star) or Summit software V5.2 (Dako).

Detection of IFN-γ was conducted by ELISA, according to BD Pharmingen's standard protocol. The capture Ab was rat anti-mouse IFN-γ (R4-6A2), and the biotinylated detection Ab was rat anti-mouse IFN-γ (XMG1.2). The OD450 was read on a SpectraMax 190 counter (Molecular Devices, Sunnyvale, Calif.) and analyzed using SOFTmax PRO 5.4 software. Graded amounts of rIFN-γ (PeproTech) were included for construction of standard curve. Cytokine concentration was extrapolated from the linear portion of the standard curve.

To inhibit PD-L1 interaction with PD-1, dendritic cell (DC) culture was supplemented with 20 mg/ml anti-PD-L1 (10F.9G2; Bio X Cell) and incubated for 2 h at 37° C. The DCs were then washed twice with PBS and used for in vitro experiments. For blockade of PD-L1 in vivo, the mice recipient of polarized BDC2.5 Th1 cells were given anti-PD-L1 Ab (200 mg/mouse) i.p. on days 1, 3, 5, and 7 post-T cell transfer. The p values were calculated using the unpaired two-tailed Student t test. ANOVA with a Bonferroni posttest was used to compare more than two groups. Prism Software v4.0c (GraphPad) was used in all statistical analyses. Data significance is denoted by *p, 0.05, **p, 0.01, and ***p, 0.001.

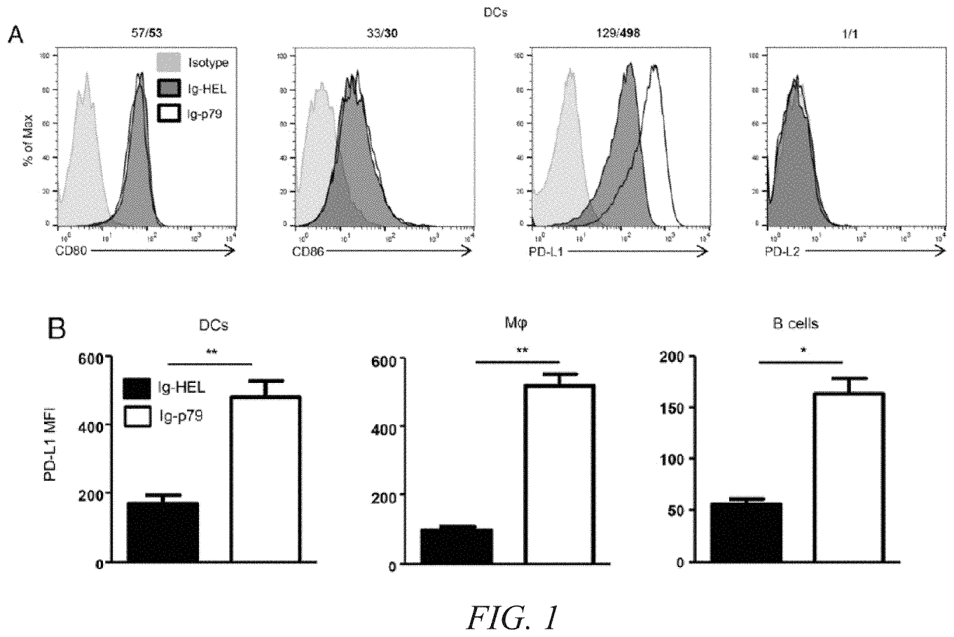

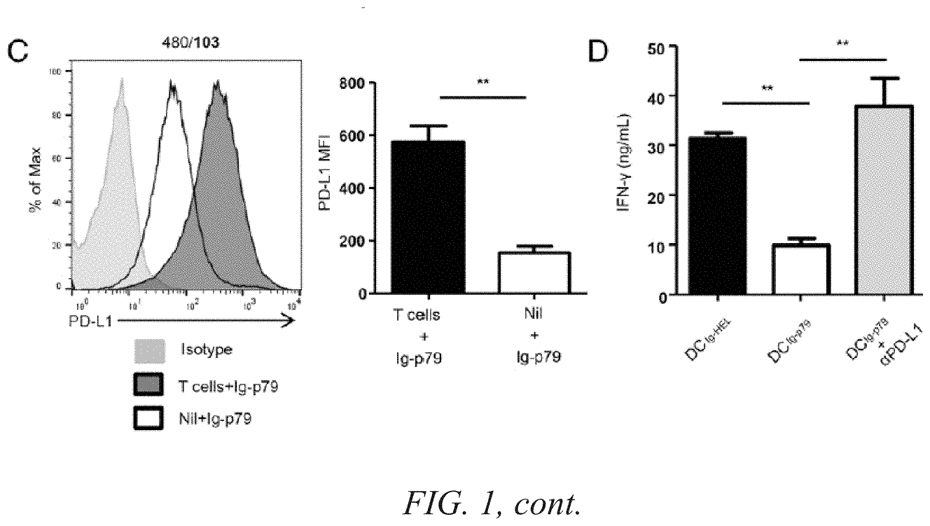

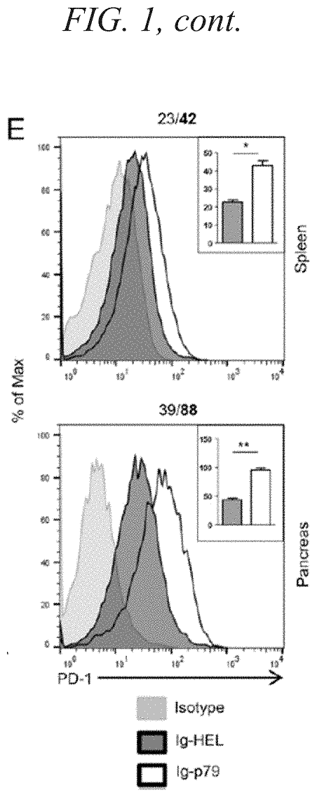

The findings indicate that only PD-L1 expression is upregulated upon treatment with Ig-p79 relative to the control Ig-HEL (FIG. 1A). This was not restricted to DCs as other APCs such as macrophages and B cells also upregulate PD-L1 in a significant manner relative to treatment with Ig-HEL (FIG. 1B). In addition, upregulation of PD-L1 on APCs requires interaction with T cells as DCs from NOD.scid mice that were not given T cell transfer before treatment with Ig-p79 could not significantly upregulate PD-L1 relative to DCs from mice given both T cell transfer and Ig-p79 treatment (FIG. 1C). The results also indicate that these interactions are Ag specific because Ig-HEL could not induce PD-L1 upregulation (FIG. 1A). The PD-L1-high (PD-L1hi) DCs from Ig-p79 treatment (DCIg-p79) display suppressive function, as coculture with naive BDC2.5 T cells in the presence of p79 peptide could not induce IFN-g production by the T cells (FIG. 1D). This suppression is dependent on PD-L1, as DC derived from Ig-HEL treatment (DCIg-HEL), which do not upregulate PD-L1, could not drive suppression of BDC2.5 T cells (FIG. 1D). Similarly, blockade of PD-L1 with anti-PD-L1 Ab during coculture of naive BDC2.5 T cells with p79-loaded DCIg-p79 could not drive suppression of IFN-g production.

Together, these findings indicate that upregulation of PD-L1 on APCs requires interaction with T cells. In addition, the PD-L1hi DCs were able to inhibit activation of BDC2.5 T cells. Because PD-L1 usually carries out its inhibitory function through interaction with PD-1 (Keir, M. E., S. C. Liang, I. Guleria, Y. E. Latchman, A. Qipo, L. A. Albacker, M. Koulmanda, G. J. Freeman, M. H. Sayegh, and A. H. Sharpe. 2006. Tissue expression of PD-L1 mediates peripheral T cell tolerance. J. Exp. Med. 203: 883-895), it was sought to determine whether T cells undergoing Ig-p79-induced tolerance upregulate PD-1 expression. This was indeed the case, as both splenic and pancreatic BDC2.5 T cells significantly upregulated PD-1 expression upon treatment with Ig-p79 relative to the control Ig-HEL (FIG. 1E).

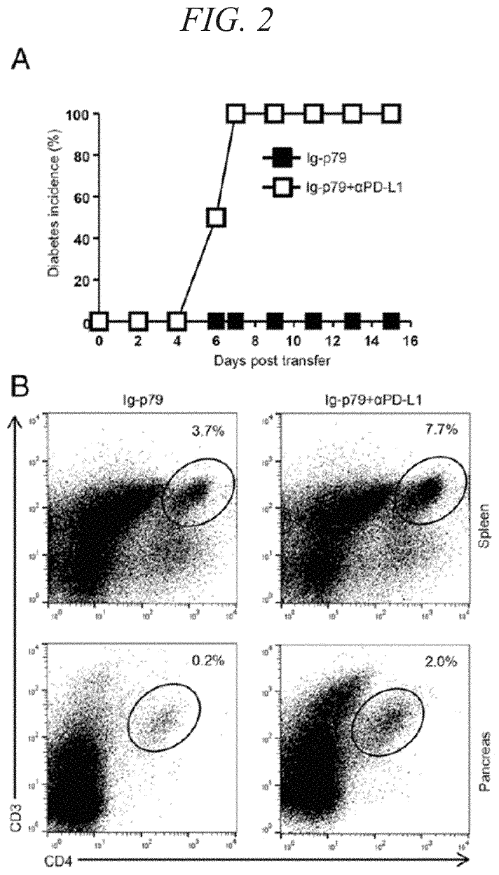

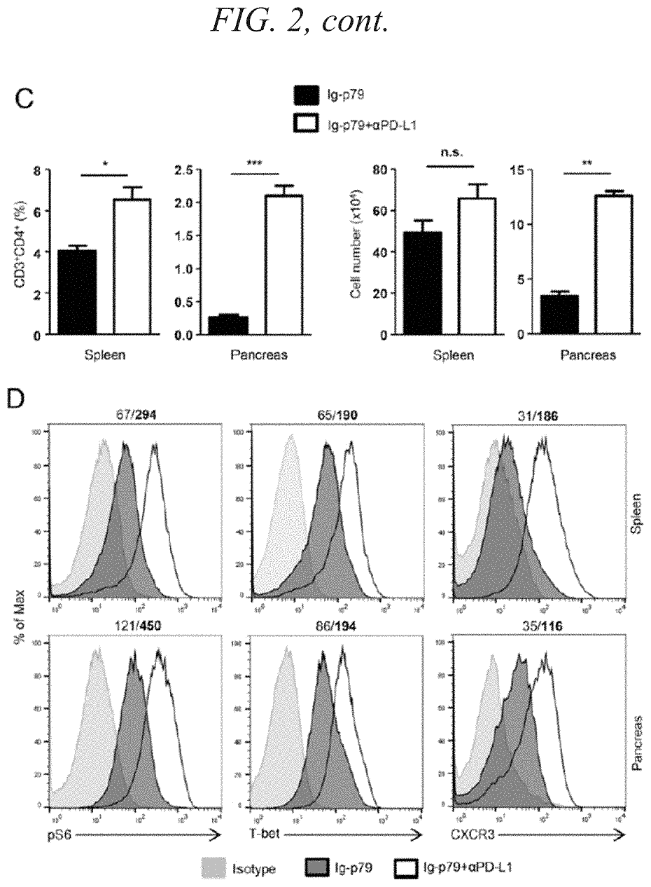

Moreover, administration of anti-PD-L1 Ab during treatment with Ig-p79 nullified suppression of T1D (FIG. 2A), and the T cells were able to reach the pancreas as their percentage increased from 0.2% during treatment with Ig-p79 alone to 2% when Ig-p79 was accompanied with anti-PD-L1 Ab (FIG. 2B). The return of the T cells to the pancreas during treatment with Ig-p79 and anti-PD-L1 Ab was significant when the percentage and absolute T cell number were compared with treatment with Ig-p79 alone (FIG. 2C). Interestingly, blockade of PD-L1 restores S6 phosphorylation and expression of T-bet and CXCR93 in both splenic and pancreatic T cells in the mice treated with Ig-p79 accompanied with anti-PD-L1 Ab, but not in animals given Ig-p79 alone (FIG. 2D). Overall, treatment with Ig-p79 induces a T cell-dependent PD-L1 upregulation on APCs, which in turn interacts with PD-1 and drives mTOC1-mediated tolerance of the T cells.

### Example 2

### Treatment of Subject with Type 1 Diabetes

From the results above it is contemplated that PD-L1 upregulated APCs could be used to treat autoimmune diseases. In one embodiment, it is contemplated that modified antigen-presenting cells (APCs) having increased expression of programmed death ligand 1 (PD-L1) can be used for treating Type 1 Diabetes (T1D). Studies are conducted to determine the effects of a composition comprising a modified APC having increased expression of PD-L1 in subjects with T1D. For example, a multicenter, randomized, double-blind, placebo-controlled study is undertaken to evaluate treatment with a weight-based or fixed dose of a composition comprising an amount of modified APC having increased expression of PD-L1 in human subjects diagnosed with T1D. More specifically, a clinical study is performed to examine the efficacy and safety of a composition comprising an amount modified APC having increased expression of PD-L1. The composition is effective to treat including, prevent, T1D.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, an additional embodiment can include placing the specimen container(s) on a surface (e.g., platform) and aspirating contents from the specimen containers. The aspirated samples can be transferred (e.g., via automation) to a spray cartridge from which the biological material can be sprayed onto substrate(s). The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

We claim:

1. A method of treating or preventing an autoimmune disease in a subject in need thereof comprising, administering to the subject an effective amount of modified antigen-presenting cells (APCs) having increased expression of programmed death ligand 1 (PD-L1).

2. The method of claim 1 wherein the increased expression of PD-L1 is induced by contacting the APC with an immunoglobulin.

3. The method of claim 2 wherein the immunoglobulin is an immunoglobulin-polypeptide chimera.

4. The method of claim 1, further comprising isolating APCs from the subject or an APC donor to obtain purified APCs.

5. The method of claim 4, further comprising contacting the purified APCs with a targeting molecule to generate modified APCs.

6. The method of claim 5 wherein the targeting molecule is an immunoglobulin-polypeptide chimera.

7. The method of claim 6 wherein the immunoglobulin-polypeptide chimera is selected from the group consisting of Ig-GAD2, Ig-GAD1, Ig-p79, Ig-INSβ and any combination thereof.

8. The method of claim 4, further comprising administering to the subject an effective amount of an immunoglobulin prior to isolating the APCs.

9. The method of claim 8, wherein the immunoglobulin is an immunoglobulin-polypeptide chimera.

10. The method of claim 1, wherein at least a portion of the increased expression of PD-L1 is from overexpression of exogenous PD-L1.

11. The method of claim 10, wherein the exogenous PD-L1 is a nucleic acid sequence encoding PD-L1.

12. The method of claim 1 wherein the APC is a dendritic cell, a macrophage, a Langerhans cell, a B cell, or a combination thereof.

13. The method of claim 1, wherein the modified APCs are characterized by one or more of the following characteristics:
(i) no change or a non-significant change in expression of at least one stimulatory molecule;
(ii) an ability to suppress inflammatory cytokine production;
(iii) an ability to increase anti-inflammatory cytokine production;
(iv) an ability to downregulate mTORR expression;
(v) an ability to inhibit activation of a T cell;
(vi) an ability to kill a T cell;
(vii) an ability to energized self-reactive T cells; and
(viii) an ability to inhibit the trafficking of self-reactive T cells, as compared to an APC that does not have increased expression of PD-L1.

14. The method of claim 13, wherein the at least one stimulatory molecule is CD80, CD86, or both.

15. The method of claim 13, wherein the inflammatory cytokine is selected from the group consisting of interleukin (IL)-1, IL-2, IL-9, IL-12, IL-17, IL-18IL-21, IL22, interferon-γ (IFN-γ, granulocyte-macrophage colony stimulating factor, and tumor necrosis factor-α (TNF-α).

16. The method of claim 13, wherein the anti-inflammatory cytokine is selected from the group consisting of IL-4, IL-5, IL-10, IL-13, IFN-α, and transforming growth factor-β (TGF-β).

17. The method of claim 1, wherein the autoimmune disease is selected from the group consisting of a cardiovascular disease, a rheumatoid disease, a glandular disease, a gastrointestinal disease, a cutaneous disease, a hepatic disease, a neurological disease, a muscular disease, a nephric disease, a disease related to reproduction, a connective tissue disease, or a systemic disease.

18. The method of claim 17, wherein the glandular disease is Type 1 diabetes.

19. A composition comprising a modified APC having increased expression of programmed death ligand 1 (PD-L1).

20. The composition of claim 19, further comprising a pharmaceutically acceptable carrier/excipient.
